(19) United States
(12) Patent Application Publication
(10) Pub. No.: US 2002/0077688 A1
(43) Pub. Date: Jun. 20, 2002

(54) Electrode-positioning body garment

(76) Inventor: Thomas C. Kirkland, Coconut Creek, FL (US)

Correspondence Address: Robert M. Downey, ROBERT M. DOWNEY, P.A., Suite 350, 150 E. Palmetto Park Road, Boca Raton, FL, 33432, US

(21) Appl. No.: 10/017,091

(22) Filed: Dec. 14, 2001

Related U.S. Application Data
(60) Provisional application No. 60/256,184, filed on Dec. 15, 2000.

(51) Int. Cl. A61N 1/00

(52) U.S. Cl. 607/142

(57) ABSTRACT

A garment made of a flexible elastic material fits snug to the body and is used in conjunction with an electrical muscle and/or nerve stimulation device. The garment is specifically structured to advantageously position electrode pads at predetermined positions on the body corresponding with different muscle or nerve groups. In one embodiment, wires connecting to the electrodes lead to an interface connector that connects to an external controller of the stimulation device. Alternatively, the entire stimulation device, including the controller, is carried on the garment. The controller enables the user to selectively direct stimulation signals to one or more of the electrode pads in order to target specific muscle or nerve groups of the body. The controller further permits selective adjustment of the duration and intensity of the signal and electrical pulse delivered to the specific muscle groups and/or nerve areas. In a further embodiment, the garment is in the form of a sleeve or wrap and is structured to cover a specific area of the body to be treated, such as the arms, legs, abdomen, chest or face.

### BACKGROUND OF THE INVENTION

1. Field of the Invention

The present invention relates generally to Transcutaneous Electrical Nerve Stimulation (TENS) and Electrical Muscle Stimulation (EMS) devices for providing a stimulating waveform and electrical pulses to select muscle groups and nerve areas of the body and, more particularly, to a partial or full body garment which includes means for positioning a plurality of electrode pads at select, predetermined locations on the body to thereby deliver electrical pulses to targeted muscle groups and/or nerve areas of the body in accordance with the needs of the user.

2. Discussion of the Related Art

TENS and EMS devices that apply stimulation signals to particular areas of the human body in order to suppress pain or stimulate a muscle are well known in the art. It has been well documented that applying a pulsed waveform, having an adjustable intensity, pulse duration and pulse width, at an inflicted area of tissue to stimulate nerve fibers is effective to suppress pain originating for the immediate area. TENS devices have been used for symptomatic relief and management of chronic, post surgical, and traumatic acute pain. EMS devices use more powerful waveforms to cause a muscle contraction. These induced muscle contractions are useful in physical therapy and body building for correcting atrophy, stimulating muscle growth, and increasing range of motion. Studies have shown that EMS stimulates large nerve axons, some of which cannot be stimulated voluntarily. It is further believed that EMS allows for additional muscle hypertrophy (i.e., increased development of tissue by enlargement, without multiplication of cells). Throughout the following specification, TENS and EMS devices will be collectively referred to as stimulation devices or units. A typical stimulation unit consists of a housing containing electrical pulse-generating circuitry and controls, a plurality of electrode pads, and wires connecting the one or more electrode pads to an interface connector. The interface connector plugs into output connectors on the stimulation unit housing.

Generally, stimulation devices include a pulse generating circuit that generates a pulse waveform having the desired parameters. One or more electrode pads positioned against a person's skin will apply the electrical pulses to the area at which the electrode is positioned. Each electrical pulse delivered by the electrode stimulates the targeted muscles, causing them to contract. By varying the frequency, amplitude, pulse width, time, and waveform shape of the stimulation signal, the effect of the stimulation on the nerve or muscle group can be controlled.

While the use of stimulation devices to perform TENS and EMS has been found to be highly effective in pain therapy, muscle rehabilitation and muscle development, the use of such devices can be inconvenient. Specifically, each time an EMS or TENS device is used, the patient must independently position each of the separate electrode pads at different locations on the body and then individually connect each of the electrode pads to the stimulation unit. After each treatment, the patient must then remove each of the electrode pads and disconnect the interconnecting wires. A further problem is encountered when trying to identify the specific location on the body for placement of the electrode pads. While doctors and physical therapists are able to more easily identify the optimal locations on the body for placement of the electrode pads, individuals using the stimulation device at home often find it difficult to identify the correct position for placement of the electrode pads on the body. Also, when trying to position electrode pads on hard to reach areas, such as on the back, many people require the assistance of another person.

Accordingly, there is an urgent need for a means for quickly and accurately positioning a plurality of electrode pads at select muscle groups and/or nerve areas for providing optimal and efficient muscle and nerve electrical stimulation treatment using a TENS or EMS device.

### SUMMARY OF THE INVENTION

The present invention is directed to a garment which may cover a portion of the body or the entire body. The garment is preferably made of a flexible, rubberized (i.e., elastomeric material) such as neoprene, spandex, lycra, etc., similar to a wetsuit or downhill skier's suit which fits snug to the body. Electrode pads are fitted to the garment at positions strategically located to correspond to particular muscle groups and/or nerve areas which can be selectively treated. Wiring from the electrode pads is routed through or between layers of material in the garment. In one or more embodiments of the invention, the wiring exits the garment and terminates at an interface. The interface, similar to a computer's serial port connection, connects to the output on a TENS unit controller or EMS unit controller to complete an electrical connection between the electrical pulse-generating circuitry of the unit and the electrode pads in the garment. Additionally, the TENS unit controller or EMS unit controller can be embodied within the garment or carried on the garment.

Wiring of the electrode pads may be color-coded to correspond with the specific areas of the body. For example, red wires may be connected to electrodes positioned on the arms, blue wires can be connected to electrodes on the legs, green wires can be connected to electrodes positioned on the abdomen muscles, orange wires can be connected to electrodes on the back, and black wires can be connected to electrodes positioned on the chest muscles. Color-coding the wires in this manner enables the user to identify the particular connection of the interface connected with the output connectors of the TENS or EMS unit. This permits operation of the TENS or EMS unit controller to deliver electrical signals to one or more selected color-coded wiring groups, thereby targeting specific muscle groups for a workout. An EMS unit used in conjunction with the garment of the present invention may be adapted to allow for selective control of electrical signal pulses. This allows each muscle group to be specifically treated in accordance with the needs of the user.

### BRIEF DESCRIPTION OF THE DRAWINGS

For a fuller understanding of the nature of the present invention, reference should be made to the following detailed description taken in conjunction with the accompanying drawings, in which:

FIG. 1 is a front perspective view of an electrode-positioning garment designed as a full body suit and having an upper jacket portion and a lower pants portion, and the garment further including an interface connector for interconnection with an external controller of an electrode muscle and/or nerve stimulation device, in accordance with one embodiment of the present invention;

FIG. 2 is a cross-sectional view taken along the line indicated as 2-2 in FIG. 1 illustrating positioning of electrode pads within the garment for contact with the skin of the user in order to deliver stimulation signals to specific muscle groups and/or nerve areas of the body;

FIG. 3 is a front elevational view showing another embodiment of the electrode-positioning garment of the present invention designed as an abdominal wrap and shown worn on a user;

FIG. 4 is a plan view of an interfacing side of the abdominal wrap of FIG. 3;

FIG. 5 is a side perspective view illustrating a further embodiment of the electrode-positioning garment of the present invention designed as a face mask and shown being worn on a user's head and covering portions of the wearer's face;

FIG. 6 is a front perspective view showing the electrode-positioning garment of the present invention in the form of a wrap or sleeve and shown fitted about the bicep of a user's arm; and

FIG. 7 is a front perspective view of the wrap or sleeve electrode-positioning garment of FIG. 6 shown worn on a user's thigh.

Like reference numerals refer to like parts throughout the several views of the drawings.

### DETAILED DESCRIPTION OF THE PREFERRED EMBODIMENTS

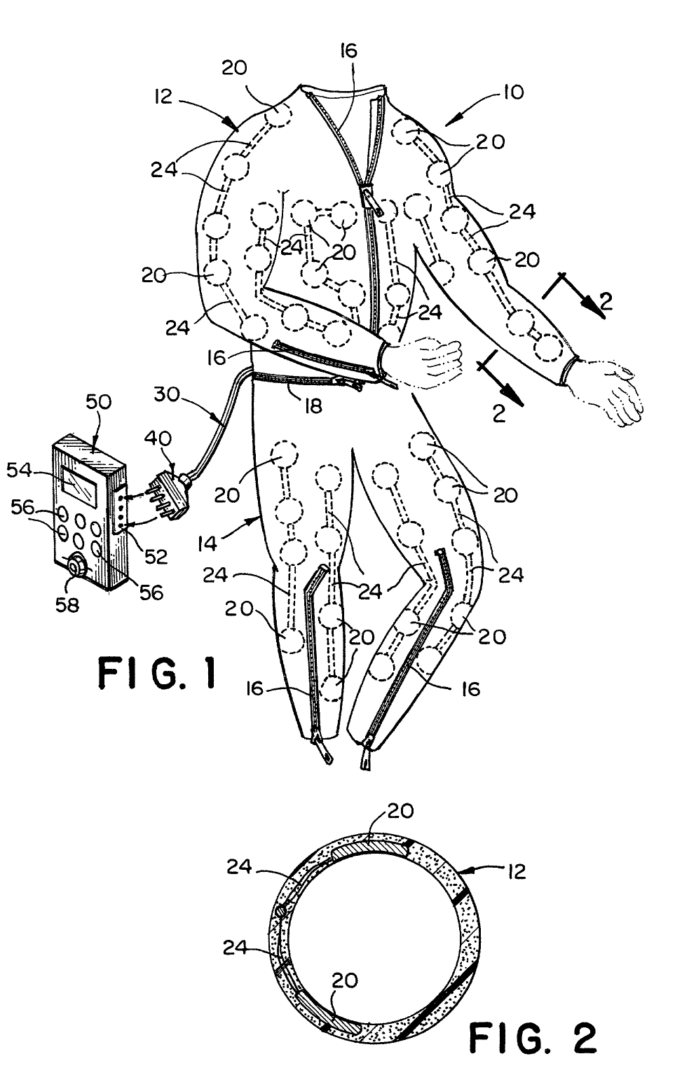

Referring to the several views of the drawings, the electrode-positioning garment is shown and is generally indicated as 10. The garment 10 is preferably made of a flexible, elastomeric material such as neoprene, spandex, lycra, etc. In one embodiment, the garment 10 is structured as a full body suit to resemble a wet suit of the type commonly used in scuba diving and surfing, as seen in FIG. 1. The suit shown in FIG. 1 may be fabricated of a thinner elastomeric material such as spandex or lycra, similar to an aerodynamic ski suit. The suit is structured to fit somewhat snug to the body, particularly at the areas of the muscle groups and/or nerves being treated.

The garment 10 is specifically structured to be used in conjunction with an electrical muscle and/or nerve stimulation device. More particularly, the garment 10 is structured to advantageously hold and position electrode pads 20 at predetermined positions on the body corresponding with specific muscle groups to be treated by electrical stimulation impulses. To accomplish this, the electrode pads 20 are placed at positions strategically located on the suit at locations which correspond to the particular muscle groups and/or nerve areas which can be selectively treated. Wiring 24 connecting to the electrode pads 20 are routed through seams on the arms, legs, and torso of the garment 10. In several embodiments, the wiring 24 exits the garment in a bundle 30 and terminates at an interface 40. The interface 40 is specifically adapted for connection with the output connectors 52 on a TENS unit or EMS unit controller 50. Controls 56 allow the user to select operation of different electrode groups corresponding with certain muscle or nerve groups of the body. The controls 56 may be color coded to identify the different electrode groups. A control knob 58 enables the adjustment of intensity and duration of electric pulses delivered to the electrode pads 20. A display 54 indicates control settings.

The garment 10 may be manufactured as a one-piece bodysuit, including an upper body portion 12 and a lower body portion 14. Alternatively, the garment 10 may be manufactured as a two-piece garment, with the upper body portion 12 being separate from the lower body portion 14. Further, it is contemplated that the garment 10 of the present invention may be structured as several pieces to cover a particular area of the body, such as one or both legs, the abdomen, the chest and/or one or both arms.

To facilitate ease of placement and removal of the garment 10 on the body, zippers 50 are provided on the arms and legs. The waist of the garment may further be provided with a zipper to permit separation of the upper body portion 12 from the lower body portion 14. Separation of the upper and lower body portions enables the user to wear either just the upper body portion or the lower body portion when targeting specific muscle groups. More particularly, the user may treat the muscle groups of the upper body during one session while wearing just the upper body portion 12 of the garment 10, and then during a different session, treat the lower muscles in the legs wearing just the lower portion 14.

The interface is specifically structured to permit selective directing of electrical signals to specific electrode pads on the garment, thereby targeting specific muscle groups. To simplify identification of the electrode pads associated with each muscle group, the controls 56 and the wires connecting between the interface and the different electrode groups may be correspondingly color-coded. Thus, for example, the wires leading to the electrodes on the legs may be color-coded blue. In this instance, operation of the blue colored control 56 will direct the pulsed signals through the blue wires and to the legs. Further, electrodes positioned on the abdomen muscles may be green, while the electrodes connecting to the chest may be colored black. Therefore, operation of the green control will activate the electrode pads positioned in the user's abdomen while operation of the black control will activate the electrode pads positioned on the user's chest. Color-coding the wires and controls in this manner also enables the user to identify the particular connection of the interface and electrode pads with the output connectors of the TENS or EMS unit. This permits operation of the TENS or EMS unit to deliver electrical signals to one or more selected color-coded wiring groups, thereby targeting specific muscle groups for stimulation. It should be noted that the EMS unit used in conjunction with the various embodiments of the garment 10 of the present invention may be adapted to allow selective control of electrical signal pulse intensity for each muscle group. By varying the duration and intensity of the electrical pulse, each muscle group can be specifically treated in accordance with the needs of the user.

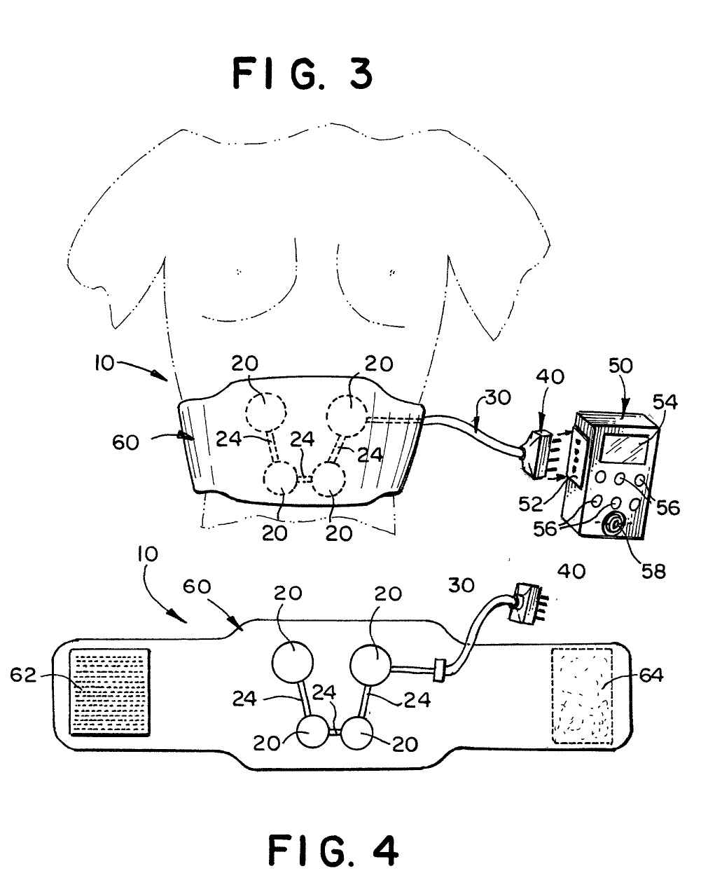

Several embodiments are shown throughout FIGS. 3-7 wherein the garment 10 is manufactured as a wrap or sleeve, which covers the specific area of the body to be treated. Specifically, FIGS. 3 and 4 illustrate an abdominal wrap 60 which is preferably formed of an elastomeric material and is structured and disposed to wrap about the lower torso of the user so that the central area of the abdominal wrap 60 covers the abdominal region of the user. The opposite ends of the abdominal wrap 60 are provided with hook and loop fasteners 62, 64 to permit releasable, adjustable fitting of the abdominal wrap 60 about the lower torso so that the central portion of the wrap is held snug against the abdominal region with the electrode pads 20 positioned against the skin of the user in electrically conductive contact therewith at the abdominal region. This particular embodiment is specifically intended to stimulate the abdominal muscles. Repeated use of the apparatus 10 in this embodiment results in tightening and firming of the abdominal region.

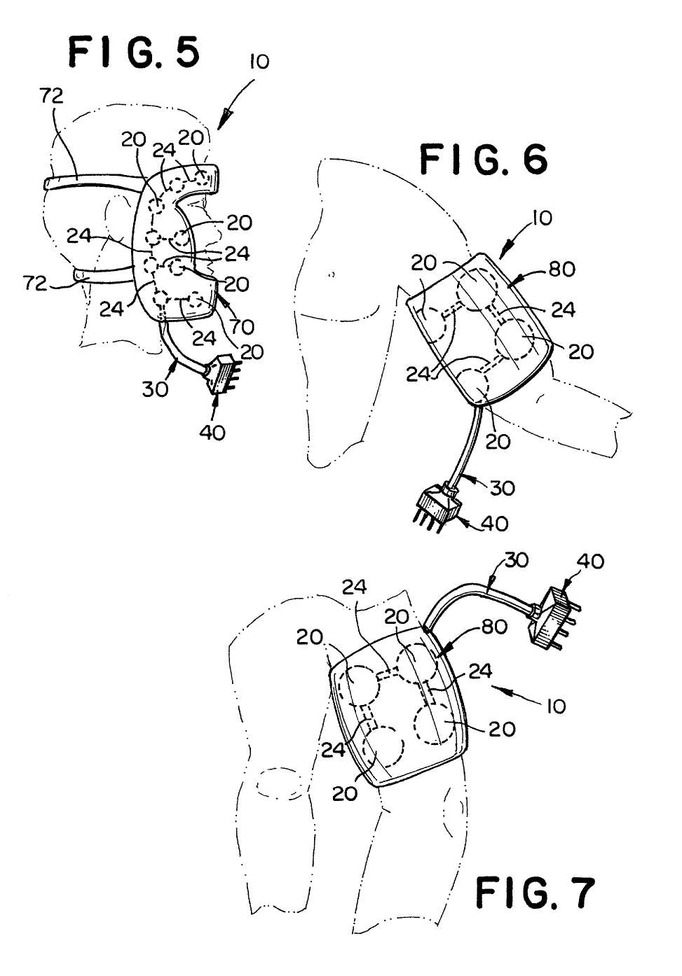

FIG. 5 illustrates yet a further embodiment of the garment 10 of the present invention. In this particular embodiment, the garment 10 is formed as a face mask 70 and is specifically structured and configured to cover portions of the user's face, including the forehead, cheeks, chin and jowls. Adjustable straps 72 are provided to secure the face mask 70 to the wearer's head so that the electrode pads 20 are positioned and held in electrically conductive contact with the skin of the face at the desired areas.

FIGS. 6 and 7 illustrate yet a further embodiment of the garment 10. In this embodiment, the garment 10 is formed as a sleeve which is adapted to fit snuggly about the user's arm or leg. Specifically, the sleeve 80 is shown in FIG. 6 fitted about the bicep of the user's arm so that the electrode pads 20 are positioned in electrically conductive contact with the skin in corresponding position with the bicep and tricep muscles of the user's arm. In FIG. 7, the sleeve 80 is shown fitted about the user's thigh so that the electrode pads 20 are positioned in electrically conductive contact with the skin and correspondingly positioned with upper leg muscles.

It should be noted that, while in the several embodiments shown throughout the drawings the interface 40 is provided for connection to a separate TENS unit or EMS unit controller 50, it is contemplated within the spirit and scope of the invention that the TENS unit or EMS unit controller 50 be incorporated within the various embodiments of the garment 10.

While the invention has been shown and described in connection with preferred embodiments thereof, it is recognized that departures from the disclosure are contemplated within the spirit and scope of the invention.

What is claimed is:

1. An apparatus for applying electrical stimulation signals to the body of a human user, comprising:
a garment structured and disposed to fit snug against the user's body so that said garment covers at least a portion of the user's body;
at least one electrode device fitted to said garment in a manner which positions said electrode device against the user's skin in electrical conducting contact therewith at a select location on the body when said garment is worn by the user;
stimulation signal means for generating the electrical stimulation signals; and
conductor means for electrically connecting said stimulation signal means to said at least one electrode device.

2. The apparatus as recited in claim 1 comprising:
a plurality of said electrode devices fitted to said garment; and
said conductor means electrically connecting said electrical signal means to each of said plurality of electrode devices.

3. The apparatus as recited in claim 2 wherein said plurality of electrode devices are arranged on said garment for positioning against the user's skin in electrical conducting contact therewith at predetermined positions on the body corresponding with specific muscle groups.

4. The apparatus as recited in claim 3 wherein said stimulation signal means includes control means for selectively directing the generated electrical stimulation signals to one or more of said plurality of electrode devices correspondingly positioned with one or more of said muscle groups.

5. The apparatus as recited in claim 4 wherein said stimulation signal means includes means for selective adjustment of the duration and intensity of the generated electrical stimulation signals.

6. The apparatus as recited in claim 5 wherein said garment is a full body suit structured and disposed to cover the upper body and lower body of the user, including the arms, chest, back, torso, hips, buttocks, and legs.

7. The apparatus as recited in claim 6 wherein said full body suit includes two pieces, including an upper body garment portion and a lower body garment portion.

8. The apparatus as recited in claim 2 wherein said garment is structured and disposed to wrap about a select area of the user's body.

9. The apparatus as recited in claim 8 wherein said garment is structured and disposed to wrap about the lower torso of the user in covering relation to the user's abdomen.

10. The apparatus as recited in claim 8 wherein said garment is structured and disposed to wrap about the user's arm.

11. The apparatus as recited in claim 8 wherein said garment is structured and disposed to wrap about the user's leg.

12. The apparatus as recited in claim 2 wherein said garment is in the form of a mask and is structured and disposed for covering at least a portion of the user's face.

13. An apparatus for applying electrical stimulation signals to the body of a human user, comprising:
a garment structured and disposed to fit snug against the user's body so that said garment covers at least a portion of the user's body;
stimulation signal means for generating the electrical stimulation signals; and
a plurality of electrode devices fitted to said garment and electrically connected to said stimulation signal means, said plurality of electrode devices each contacting the user's skin in electrical conducting contact therewith when said garment is worn by the user.

14. The apparatus as recited in claim 13 wherein said plurality of electrode devices are arranged on said garment for positioning against the user's skin in electrical conducting contact therewith at predetermined positions on the body corresponding with specific muscle groups.

15. The apparatus as recited in claim 14 wherein said stimulation signal means includes control means for selectively directing the generated electrical stimulation signals to one or more of said plurality of electrode devices to selectively stimulate one or more of the specific muscle groups.

16. The apparatus as recited in claim 15 wherein said stimulation signal means includes means for selective adjustment of the duration and intensity of the generated electrical stimulation signals.

17. The apparatus as recited in claim 16 wherein said garment is a full body suit structured and disposed to cover the upper body and lower body of the user, including the arms, chest, back, torso, hips, buttocks, and legs.

18. The apparatus as recited in claim 13 wherein said garment is structured and disposed to wrap about a select area of the user's body.

19. The apparatus as recited in claim 13 wherein said garment is structured and disposed to wrap about the lower torso of the user in covering relation to the user's abdomen.

20. The apparatus as recited in claim 13 wherein said garment is structured and disposed to wrap about the arm or leg of the user.
